(19) United States
(12) Patent Application Publication
(10) Pub. No.: US 2002/0090349 A1
(43) Pub. Date: Jul. 11, 2002

(54) Process for the biocidal treatment of surface

(76) Inventors: Vance Bergeron, Lyon (FR); Jean-Pierre Marchand, Lyon (FR); Harold Schoonbrood, Victoria (AU)

Correspondence Address: RHODIA INC., CN-7500, 259 Prospect Plains Road, CRANBURY, NJ, 08512, US

(21) Appl. No.: 10/060,823

(22) Filed: Jan. 30, 2002

Related U.S. Application Data
(62) Division of application No. 09/350,240, filed on Jul. 9, 1999.
(60) Provisional application No. 60/092,124, filed on Jul. 9, 1998.

(51) Int. Cl. A61L 9/00; A61K 7/06

(52) U.S. Cl. 424/70.11; 422/32

(57) ABSTRACT

Process for the biocidal treatment of surfaces, by applying an aqueous biocidal composition containing a biocidal agent (B), a surfactant (SA) when the said biocide is hydrophobic, and at least one water-soluble or water-dispersible organic copolymer (C) comprising at least one oligomeric or macromolecular unit (D) which can interact with the said biocide or with the micelles of surfactant(s) containing the said biocide when it is hydrophobic, and at least one hydrophilic macromolecular unit (E) which can interact with the surface to be treated and optionally with the said biocide. Use of the copolymer (C) in a biocidal composition for the treatment of surfaces, as an agent for the vectorization and/or controlled release of the said biocide onto the surface to be treated.

The present invention relates to a process for the biocidal treatment of cutaneous, keratinous or textile surfaces, as well as of hard industrial, domestic or community surfaces, using an aqueous biocidal composition based on a biocide, at least one water-soluble or water-dispersible copolymer and at least one surfactant when the said biocide is hydrophobic. The invention is also directed towards the use, in an aqueous biocidal composition for the treatment of cutaneous, keratinous or textile surfaces, as well as of hard industrial, domestic or community surfaces, containing a biocide and at least one surfactant when the said biocide is hydrophobic, of a water-soluble or water-dispersible copolymer, as an agent for the vectorization and/or controlled release of the said biocide onto the surface to be treated. The invention also relates to aqueous biocidal compositions in which the said biocidal agent is cationic or non-cationic.

A first subject of the invention consists of a process for the biocidal treatment of cutaneous, keratinous or textile surfaces, as well as of hard industrial, domestic or community surfaces, by applying to the said surfaces an aqueous biocidal composition comprising:

at least one biocidal agent (B)

at least one surfactant (SA) when the said biocide is hydrophobic, and

at least one water-soluble or water-dispersible organic copolymer (C) comprising

at least one oligomeric or macromolecular unit (D) which can interact with the said biocide or with the micelles of surfactant(s) containing the said biocide when the latter is hydrophobic, and

at least one hydrophilic macromolecular unit (E) which can interact with the surface to be treated and optionally with the said biocide.

The phenomena of interaction between the unit(s) (D) and the surfactant micelles, or of interaction between the unit(s) (E) and the surface to be treated can be of various natures. They can be, for example, electrostatic interactions, dipolar interactions, formation of hydrogen bonds or hydrophobic interactions.

The said copolymer (C) comprising one or more units (D) and one or more units (E) can be a block copolymer or a grafted copolymer.

It can have a molecular mass of about 10,000 to 10,000,000, preferably of about 50,000 to 2,000,000.

When it is a block copolymer, each unit (D) or (E) represents a block of the said copolymer.

When the copolymer (C) is a grafted copolymer, it can consist:

of a trunk comprising an oligomeric or macromolecular unit (D) and of one or more grafts comprising a macromolecular unit (E)

or alternatively of a trunk comprising a macromolecular unit (E) and of one or more grafts comprising an oligomeric or macromolecular unit (D).

Preferably, the said copolymer (C) is a grafted copolymer consisting of a trunk comprising an oligomeric or macromolecular unit (D) and one or more grafts comprising a macromolecular unit (E).

Among the oligomeric or macromolecular units which can constitute the unit(s) (D), mention may be made of those derived from:

oxyalkylene oligomers or polymers (D1), in which the oxyalkylene repeating units are the same or different, the alkylene residue being linear or branched and containing from 2 to 4 carbon atoms, the said oligomers or polymers having an overall degree of polymerization of 2 to 3000, preferably of 5 to 500.

When the oxyalkylene repeating units are different, they can be distributed randomly or preferably in blocks.

As examples of oxyalkylene oligomers or polymers (D1), mention may be made of polyoxyethylene (POE) monoblocks, polyoxyethylene-polyoxypropylene (POE-POP) diblocks and (POE-POP-POE) triblocks

vinylpyrrolidone polymers or copolymers (D2) having a molecular mass of about 5000 to 1,000,000, preferably of 5000 to 400,000; they can contain up to 50 mol % of units derived from at least one comonomer such as N-vinylimidazole or vinyl acetate,

polyvinyl alcohols (D3) having a molecular mass of about 5000 to 1,000,000, preferably of 5000 to 400,000.

Among the macromolecular units which can constitute the hydrophilic unit (E), mention may be made of those derived from:

homopolymers or copolymers (E1) obtained from the polymerization of at least one anionic, amphoteric or cationic, water-soluble, ethylenically unsaturated monomer. Among the anionic water-soluble ethylenically unsaturated monomers, mention may be made of water-soluble ethylenically unsaturated carboxylic acids, in particular acrylic, methacrylic, fumaric, maleic or itaconic acid or anhydride, N-methacroylalanine, N-acryloylhydroxyglycine, etc. or water-soluble salts thereof, sulfonated or phosphonated water-soluble ethylenically unsaturated monomers such as, in particular, sulfopropyl acrylate or water-soluble salts thereof, water-soluble styrene sulfonates, or vinylphosphonic acid and water-soluble salts thereof.

Among the amphoteric water-soluble ethylenically unsaturated monomers, mention may be made in particular of N,N-dimethyl-N-methacryloxyethyl-N-(3-sulfopropyl)ammonium sulfobetaine, methacrylic acid amidopropyldimethylammonium betaine, 1-vinyl-3-(3-sulfopropyl)imidazolidium betaine, and 2-vinylpyridinium sulfopropylbetaine.

Among the cationic water-soluble ethylenically unsaturated monomers, mention may be made in particular of trimethylaminoethyl-acrylamide or -methacrylamide chloride or bromide, trimethylaminobutyl-acrylamide or -methacrylamide methylsulfate, trimethylaminopropyl methacrylate chloride and 1-ethyl-2-vinylpyridinium bromide, chloride or methylsulfate.

copolymers (E2) which are preferably water-soluble, obtained from the copolymerization of at least one water-soluble, anionic, amphoteric or cationic, preferably anionic, ethylenically unsaturated monomer and of at least one nonionic, preferably hydrophilic ethylenically unsaturated comonomer.

Among these nonionic comonomers, mention may be made of hydroxyethyl acrylate, hydroxyethyl methacrylate, methoxyethyl acrylate, acrylamide, N-dimethylaminomethyl methacrylate, methyl or ethyl acrylate or methacrylate, vinyl acetate, methyl or ethyl vinyl ether, N-vinylpyrrolidone, styrene, vinyl chloride and acrylonitrile.

The amount of units derived from the said comonomer(s) can represent up to 70%, preferably up to 50%, of the weight of the said copolymer (E2).

The said copolymers (E1) and (E2) are preferably random. The hydrophilic unit (E) can most preferably be obtained from the polymerization of at least one water-soluble ethylenically unsaturated carboxylic acid (or a salt) and optionally of at least one water-soluble amphoteric ethylenically unsaturated monomer; the amount of amphoteric ethylenically unsaturated monomer can represent up to 50%, for example from 1 to 50% and preferably from about 2 to 20%, of the total weight of ethylenically unsaturated monomers.

The copolymers (C) can be obtained according to the known techniques for the preparation of block or grafted copolymers.

Thus, the block copolymers can be obtained by covalent coupling of a polymer containing the unit (D) and a polymer containing the unit (E), using groups such as amide, ester, ether, thioester, thioether, urea, urethane, amine or others resulting from the reaction of the terminal functions of the two types of polymer, as described in WO 95/24430. They can also be obtained by radical polymerization of one or more ethylenically unsaturated monomer(s) from which the unit (E) is derived, in the presence of a polymer containing the unit (D) having a chain end functionalized with a xanthate or thiuram unit, as described in EP-A-539,256, EP-A-342,073 and EP-A-418,116.

Grafted copolymers in which the trunk comprises a macromolecular unit (E) and one or more grafts containing an oligomeric or macromolecular unit (D) can be obtained:

by radical copolymerization of an ethylenically unsaturated macromonomer containing the unit (D) and one or more ethylenically unsaturated monomer(s) from which the unit (E) is derived, as described in EP-A-583,814, EP-A-182,523 or EP-A-522,668.

by partial reaction of the pendant reactive units of a polymer containing the unit (E) with the reactive terminal functions of an oligomer or of a polymer containing the unit (D), as described in EP-A-182,523, EP-A-522,668 and WO 95/24430.

Grafted copolymers in which the trunk comprises an oligomeric or macromolecular unit (D) and one or more grafts containing a macromolecular unit (E) can be obtained by radical polymerization or irradiation, in aqueous phase or in bulk, of the monomer(s) from which the unit (E) is derived in the presence of an oligomer, polymer or copolymer from which the unit (D) is derived. This technique is described, for example, in WO 97/00275.

The hydrophilic unit (E) can have a molecular mass preferably of greater than 10,000. This can be up to 10,000,000.

The relative amounts of unit(s) (D) and of unit(s) (E) can correspond to a ratio of the total mass of units (D)/total mass of units (E) of about 5/100 to 50/100, preferably of about 10/100 to 30/100.

As examples of copolymer (C), mention may be made most particularly of:

grafted copolymers with a molecular mass of about 10⁴ to 10⁷, having a trunk consisting of a polyoxyethylene block with a molecular mass of about 10² to 2×10⁵, and of grafts consisting of polyacrylic acid,

grafted copolymers with a molecular mass of about 10⁴ to 10⁷, having a trunk consisting of a polyoxyethylene-polyoxypropylene diblock with a molecular mass of about 10² to 2×10⁵, and of grafts consisting of polyacrylic acid,

grafted copolymers with a molecular mass of about 10⁴ to 10⁷ having a trunk consisting of a polyoxyethylene-polyoxypropylene diblock with a molecular mass of about 10² to 2×10⁵, and of grafts consisting of random units derived from acrylic acid and from hydroxyethyl acrylate,

grafted copolymers with a molecular mass of about 10⁴ to 10⁷, having a trunk consisting of a polyoxyethylene block with a molecular mass of about 10² to 2×10⁵, and of grafts consisting of random units derived from acrylic acid and from N,N-dimethyl-N-(2-ethyl methacrylate)-N-(3-sulfopropyl)ammonium sulfobetaine,

grafted copolymers with a molecular mass of about 10⁴ to 10⁷, having a trunk consisting of a polyvinyl-pyrrolidone block with a molecular mass of about 5×10³ to 10⁶, and of grafts consisting of polyacrylic acid,

grafted copolymers with a molecular mass of about 10⁴ to 10⁷, having a trunk consisting of a random copolymer unit containing acrylic acid and methacrylate units, and of grafts consisting of methoxy polyethylene glycol units with a molecular mass of about 10² to 2×10⁵.

The biocidal agents which can be used in order to carry out the biocidal treatment process according to the invention can be hydrophilic or hydrophobic. A biocidal agent is considered, according to the invention, as being “hydrophobic” when its solubility in water at 25° C. is less than 1% by weight, preferably less than 0.1% by weight.

The choice of the nature of the biocidal agent(s) used depends on the desired application (body hygiene, disinfection of textiles or of various hard surfaces).

As examples of hydrophilic biocidal agents (B), mention may be made of

cationic biocides such as

quaternary monoammonium salts such as

cocoalkylbenzyldimethylammonium, (C₁₂-C₁₄)alkylbenzyldimethylammonium, cocoalkyldichlorobenzyldimethylammonium, tetradecylbenzyldimethylammonium, didecyldimethylammonium or dioctyldimethylammonium chlorides,

myristyltrimethylammonium or cetyltrimethylammonium bromides

monoquaternary heterocyclic amine salts such as laurylpyridinium, cetylpyridinium or (C₁₂-C₁₄)alkylbenzylimidazolium chlorides

triphenylphosphonium fatty alkyl salts such as myristyltriphenylphosphonium bromide

polymeric biocides, such as those derived from the reaction

of epichlorohydrin and of dimethylamine or of diethylamine

of epichlorohydrin and of imidazole

of 1,3-dichloro-2-propanol and of dimethylamine

of 1,3-dichloro-2-propanol and of 1,3-bis(dimethylamino)-2-propanol

of ethylene dichloride and of 1,3-bis(dimethylamino)-2-propanol

of bis(2-chloroethyl) ether and of N,N′-bis(dimethylaminopropyl)-urea or -thiourea

biguanidine polymeric hydrochlorides, such as Vantocil IB

amphoteric biocides such as derivatives of N-(N′-C₈-C₁₈alkyl-3-aminopropyl) glycine, of N-(N′-(N″-C₈-C₁₈alkyl-2-aminoethyl)-2-aminoethyl)glycine, of N,N-bis(N′-C₈-C₁₈alkyl-2-aminoethyl)glycine, such as (dodecyl)(aminopropyl)glycine and (dodecyl)(diethylenediamine)glycine

amines such as N-(3-aminopropyl)-N-dodecyl-1,3-propanediamine

halogenated biocides, for instance iodophores and hypochlorite salts, such as sodium dichloroisocyanurate

phenolic biocides such as:

phenol, resorcinol and cresols.

As examples of hydrophobic biocidal agents (B), mention may be made of

para-chloro-meta-xylenol or dichloro-meta-xylenol

4-chloro-m-cresol

resorcinol monoacetate

mono- or poly-alkyl or -aryl phenols, cresols or resorcinols, such as o-phenylphenol, p-tert-butylphenol or 6-n-amyl-m-cresol

alkyl and/or aryl-chloro- or -bromophenols, such as o-benzyl-p-chlorophenol

halogenated diphenyl ethers such as 2′,4,4′-trichloro-2-hydroxy-diphenyl ether (triclosan) and 2,2′-dihydroxy-5,5′-dibromo-diphenyl ether

chlorophenesin (p-chloro-phenylglyceric ether).

Among the surfactants which can be present, mention may be made of those of nonionic, anionic, amphoteric or zwitterionic type.

Mention may be made in particular of:

anionic surfactants such as alkyl sulfates, alkyl sulfonates, alkylbenzene sulfonates, soaps, alkyl ether carboxylates, alkyl isethionates, N-acyl-N-alkyl taurates, alkyl phosphates, alkyl sulfosuccinates and sulfonated derivatives of fatty acids, of alkali metals

nonionic surfactants such as ethylene oxide propylene oxide block polymers, polyethoxylated sorbitan esters, sorbitan fatty esters, ethoxylated fatty esters (containing from 1 to 25 ethylene oxide units), polyethoxylated C₈-C₂₂ alcohols (containing from 1 to 25 ethylene oxide units), polyethoxylated C₆-C₂₂ alkylphenols (containing from 5 to 25 ethylene oxide units), alkylpolyglycosides, amine oxides (such as C₁₀-C₁₈ alkyldimethylamine oxides and C₈-C₂₂ alkoxyethyldihydroxyethylamine oxides)

amphoteric or zwitterionic surfactants such as (C₆-C₂₀)alkyl amphoacetates or amphodiacetates (such as cocoamphoacetates), C₁₀-C₁₈ alkyldimethylbetaines, C₁₀-C₁₈ alkylamidopropyldimethylbetaines, C₁₀-C₁₈ alkyldimethylsulfobetaines or C₁₀-C₁₈ alkylamidopropyldimethylsulfobetaines.

The aqueous biocidal composition which can be used to carry out the process of the invention can contain:

from about 0.01 to 20%, preferably from about 0.03 to 5%, of its weight of at least one noncationic biocidal agent (B)

from about 0.1 to 50%, preferably from about 0.5 to 30%, of its weight of at least one surfactant (SA), when the said biocidal agent is hydrophobic, and

from about 0.005 to 20%, preferably from about 0.05 to 10%, most particularly from 0.05 to 5%, of its weight of at least one copolymer (C).

The amount of biocidal agent (B) to be used depends on the field of application for which the process of the invention is used.

Thus, in the field of body hygiene for the hair or the skin, the amount of biocide (B) used is generally from about 0.05 to 0.3% in order to satisfy the regulations in force.

If desired, a solubilizing agent can be present in the said composition in order to facilitate the solubilization of the said biocide (B), when the latter is hydrophobic. As examples of solubilizing agents, mention may be made of alcohols such as ethanol, isopropanol, propylene glycol or other solvents.

The biocidal composition used to carry out the process of the invention can be of different type, which depends on the field of application for which the process of the invention is used.

It can be a body hygiene composition for the hair or the skin, in particular in the form of shampoos, lotions, shower gels for the hair or the body, or liquid soaps for the face or the body.

It can also be a detergent composition, in particular for handwashing laundry, for rinsing laundry and for washing up by hand.

They can also be detergent compositions for cleaning and disinfecting hard industrial, domestic or community surfaces.

According to the invention, besides the biocide, the optional surfactant(s) and the copolymer (C) which are the main constituents of the aqueous biodical composition, other constituents may be present, the nature of which depends on the field of application for which the process of the invention is used.

Thus, biocidal compositions for the treatment of hard surfaces may also comprise additives such as chelating agents [such as aminocarboxylates (ethylenediaminetetraacetates, nitrilotriacetates, N,N-bis(carboxymethyl)glutamates, citrates)], alcohols (ethanol, isopropanol, glycols), detergent adjuvants (phosphates, silicates), surfactants, dyes, fragrances or the like. Compositions for hair and skin hygiene can also contain surfactants, wetting agents, emollients, viscosity-modifying agents, gelling agents, sequestering agents, conditioners, dyes, fragrances or the like.

Compositions for washing up by hand can also contain surfactants, viscosity-modifying agents, hydrotropic agents, fragrances, dyes or the like.

Compositions for handwashing laundry can also contain surfactants, inorganic or organic detergent adjuvants, anti-soiling agents, anti-redeposition agents, chelating agents, dispersing polymers, softeners, enzymes, fragrances or the like.

The said process for the biocidal treatment of surfaces, which forms the subject of the invention, can be used in order to carry out the treatment of keratinous or cutaneous body surfaces, the surfaces of textile articles, various hard surfaces such as floor tiles, floorboards, walls, working surfaces, equipment, furniture, instruments, etc. in industry, the agrifood field, domestic fields (kitchens, bathrooms, toilets, etc.) and in the community sector, as well as for washing up.

Among the hard surfaces which can be treated, mention may be made in particular of those made of ceramic, porcelain, glass, polyvinyl chloride, formica or any other hard organic polymer, stainless steel, aluminium, wood, etc.

The disinfection operation consists in applying or placing the said aqueous biocidal composition, optionally diluted from 1 to 1000 times, preferably from 1 to 100 times, in contact with the surface to be treated.

The amount of aqueous biocidal composition which can favourably be used is that corresponding to a deposition of 0.01 to 10 g, preferably of 0.1 to 1 g, of biocide per m² of surface and to a deposition of 0.001 to 2 g, preferably of 0.01 to 0.5 g, of copolymer (C) per m² of surface.

Among the microorganisms whose proliferation can be controlled by use of the process of the invention, mention may be made of

Gram-negative bacteria such as: Pseudomonas aeruginosa; Escherichia coli; Proteus mirabilis

Gram-positive bacteria such as: Staphylococcus aureus; Streptococcus faecium

other dangerous bacteria in food, such as: Salmonella typhimurium; Listeria monocytogenes; Campylobacter jejuni; Yersinia enterocolitica

yeasts, such as: Saccharomyces cerevisiae; Candida albicans

fungi, such as: Aspergillus niger; Fusarium solani; Pencillium chrysogenum

algae, such as: Chlorella saccharophilia; Chlorella emersonii; Chlorella vulgaris; Chlamydomonas eugametos.

Another subject of the invention consists of the use,

in an aqueous biocidal composition for the biocidal treatment of cutaneous, keratinous or textile surfaces, as well as of hard industrial, domestic or community surfaces, containing at least one biocidal agent (B) and at least one surfactant (SA) when the said biocidal agent is hydrophobic, of at least one water-soluble or water-dispersible organic copolymer (C) as an agent for the vectorization and/or controlled release of the said biocide(s) onto the hard surface to be treated, the said copolymer (C) comprising

at least one oligomeric or macromolecular unit (D) which can interact with the said biocide or with the micelles of surfactant(s) containing the said biocide when the latter is hydrophobic, and

at least one hydrophilic macromolecular unit (E) which can interact with the surface to be treated and optionally with the said biocide.

The nature or examples of the various constituents of the said biocidal composition, of the copolymer (C), and likewise the amounts of these various constituents and of copolymer (C) present in the said composition have already been mentioned above.

Another subject of the invention consists of an aqueous biocidal composition containing

at least one noncationic biocidal agent (B)

at least one surfactant (SA) when the said biocide is hydrophobic, and

at least one water-soluble or water-dispersible organic copolymer (C) comprising

at least one oligomeric or macromolecular unit (D) which can interact with the said biocide or with the micelles of surfactant(s) containing the said biocide when the latter is hydrophobic, and

at least one hydrophilic macromolecular unit (E) which can interact with the surface to be treated and optionally with the said biocide.

Along with the noncationic biocide (B), the optional surfactant(s) (SA) and the copolymer (C), which are the main constituents of the aqueous biocidal composition, other constituents may be present, the nature of which depends on the field of application of the said biocidal composition.

Examples of noncationic biocides (B), of optional surfactant(s) (SA) and of copolymer (C), and likewise of the amounts of these various constituents presented in the said aqueous biocidal composition have already been mentioned above. Similarly, the nature of the other optional constituents has already been mentioned above.

The subject of the present invention is also an aqueous biocidal composition containing

at least one cationic biocidal agent (B)

at least one surfactant (SA) when the said biocide is hydrophobic, and

at least one water-soluble or water-dispersible organic copolymer (C) chosen from block or grafted copolymers comprising

at least one oligomeric or macromolecular unit (D) chosen from those derived from

oxyalkylene oligomers or polymers (D1) in which the oxyalkylene repeating units are the same or different, the alkylene residue being linear or branched and containing from 2 to 4 carbon atoms, the said oligomers or polymers having an overall degree of polymerization of 2 to 3000, preferably of 5 to 500

vinylpyrrolidone polymers or copolymers (D2) having a molecular mass of about 5000 to 1,000,000, preferably of 5000 to 400,000; they can contain up to 50 mol % of units derived from at least one comonomer such as N-vinylimidazole or vinyl acetate

polyvinyl alcohols (D3) having a molecular mass of about 5000 to 1,000,000, preferably of 5000 to 400,000,

and at least one hydrophilic macromolecular unit (E) chosen from those derived from

homopolymers or copolymers (E1) obtained from the polymerization of at least one anionic, amphoteric or cationic, water-soluble, ethylenically unsaturated monomer

copolymers (E2), which are preferably water-soluble, obtained from the copolymerization of at least one water-soluble, anionic, amphoteric or cationic, preferably anionic, ethylenically unsaturated monomer and of at least one nonionic, hydrophilic, ethylenically unsaturated comonomer, each oligomeric or macromolecular unit (D) representing a block of the copolymer (C) when the latter is a block copolymer, or the trunk of the copolymer (C) when the latter is a grafted copolymer, and each hydrophilic macromolecular unit (E) representing a block of the copolymer (C) when the latter is a block copolymer, or the graft(s) of the copolymer (C) when the latter is a grafted copolymer.

Examples of noncationic biocide (B), of optional surfactant(s) (SA), of units (D1), (D2), (D3), (E1) and (E2) contained in the copolymer (C), of copolymer (C) and likewise the amounts of these various constituents present in the said biocidal composition have already been mentioned above. Similarly, the nature of the other optional constituents has already been mentioned above.

In this specification, all parts, percentages, ratios, averages and the like are by weight, unless otherwise apparent to one of ordinary skill from the context of use.

The following examples are given by way of illustration.

The molecular mass of the grafted copolymer can be measured by Gel Permeation Chromatography under the following conditions: four (4) columns TSK gel; eluent of water/acetonitrile 80/20 vol % plus 0.1 M sodium nitrate plus 150 ppm sodium azide; and flow-rate 1 ml/mn. The results should be weight average molecular mass expressed as polyoxyethylene equivalents.

### EXAMPLE 1

Grafted copolymer consisting of a polyoxyethylene trunk and of polyacrylic acid grafts

10 g of sodium hydroxide are dissolved in 680 g of water, at room temperature, in a 2-liter glass reactor fitted with a jacket and a stirring system (190 rev/min). The temperature is then increased to 65° C.

The following are prepared:

an initiator solution comprising 0.105 g of ammonium persulfate and 30 g of water (Solution 1)

a solution comprising 30 g of water and 70 g of acrylic acid (Solution 2)

and a solution comprising 10 g of sodium hydroxide and 20 g of water (Solution 3).

14 g of polyethylene oxide (molecular mass of 20,000 g/mol) are dissolved in the sodium hydroxide solution at 65° C. contained in the reactor.

The three solutions are then introduced simultaneously and continuously into the said reactor at 65° C., Solutions 2 and 3 over a period of 120 minutes and Solution 1 over a period of 150 minutes.

Once the introductions are complete, the reaction mixture is left stirring for 180 minutes at 65° C. The mixture is finally cooled to room temperature.

A solution of grafted copolymer with a molecular mass of about 600,000 is obtained.

### EXAMPLE 2

Grafted copolymer consisting of a polyacrylic acid/methacrylate random copolymer trunk and of polyoxyethylene grafts

586.7 g of water are introduced, at room temperature, into a 2-liter glass reactor fitted with a jacket and a stirring system (190 rev/min). The reactor temperature is then increased to 50° C.

The following are prepared:

an initiator solution comprising 0.20 g of ammonium persulfate and 30 g of water (Solution 1), this solution being introduced in a single portion into the reactor when the temperature of the latter reaches 50° C. (time t0).

a solution comprising 30 g of water and 100 g of acrylic acid (Solution 2)

and a solution comprising 0.1 g of sodium disulfite and 20 g of water (Solution 3).

Solutions 2 and 3 are introduced continuously into the reactor at time t0, over 120 minutes, along with 33.3 g of methoxy polyethylene glycol methacrylate (molecular mass 350 g/mol).

Once the introductions are complete, the reaction mixture is left stirring for 360 minutes at a temperature of 50° C.

The mixture is finally cooled to room temperature.

A solution of grafted copolymer is obtained.

### EXAMPLE 3

Mixture of a grafted copolymer consisting of a POE-POP diblock trunk and polyacrylic acid crafts and of a crafted copolymer consisting of a polyvinylpyrrolidone trunk and polyacrylic acid grafts

10 g of sodium hydroxide are dissolved in 717 g of water, at room temperature, in a 2-liter glass reactor fitted with a jacket and a stirring system (120 rev/min). The temperature is then increased to 65° C.

The following are prepared:

an initiator solution comprising 0.21 g of ammonium persulfate and 10 g of water (Solution 1),

a solution comprising 30 g of water and 70 g of acrylic acid (Solution 2)

and a solution comprising 10 g of sodium hydroxide and 20 g of water (Solution 3).

10.5 g of polyvinylpyrrolidone (PVP K30) and 10.5 g of a diblock copolymer of ethylene oxide and of propylene oxide (molecular mass 4150 g/mol, 50% by weight EO and 50% by weight PO) are dissolved in the sodium hydroxide solution at 65° C. contained in the reactor.

After Solution 1 has been introduced, in a single portion, into the reactor, Solutions 2 and 3 are introduced continuously over 120 minutes.

Once the introductions are complete, the reaction mixture is left stirring for 300 minutes at 65° C.

The mixture is finally cooled to room temperature.

A solution of a mixture of grafted copolymers is obtained.

### EXAMPLE 4

Grafted copolymer consisting of a POE-POP diblock trunk and of acrylic acid/2-hydroxyethyl acrylate random copolymer grafts

10 g of sodium hydroxide are dissolved in 610 g of -water, at room temperature, in a 2-liter glass reactor fitted with a jacket and a stirring system (120 rev/min). The temperature is then increased to 65° C.

The following are prepared:

an initiator solution comprising 0.21 g of ammonium persulfate and 10 g of water (Solution 1),

a solution comprising 186 g of water, 14 g of 2-hydroxyethyl acrylate and 70 g of acrylic acid (Solution 2)

and a solution comprising 10 g of sodium hydroxide and 20 g of water (Solution 3).

14 g of a diblock copolymer of ethylene oxide and of propylene oxide (molecular mass 4150 g/mol, 50% by weight EO and 50% by weight PO) are dissolved in the sodium hydroxide solution at 65° C. contained in the reactor.

After Solution 1 has been introduced, in a single portion, into the reactor, Solutions 2 and 3 are introduced continuously over 120 minutes.

Once the introductions are complete, the reaction mixture is left stirring for 300 minutes at 65° C.

The mixture is finally cooled to room temperature.

A solution of grafted copolymer is obtained.

### EXAMPLE 5

Grafted copolymer consisting of a polyethylene oxide trunk and of acrylic acid/N,N-dimethyl-N-(2-ethyl methacrylate)-N-(3-sulfopropyl)ammonium sulfobetaine random copolymer crafts

60 g of sodium hydroxide are dissolved in 3900 g of water, at room temperature, in a 6-liter glass reactor fitted with a jacket and a stirring system (120 rev/min). The temperature is then increased to 65° C.

The following are prepared:

an initiator solution comprising 1.26 g of ammonium persulfate and 50 g of water (Solution 1)

a solution comprising 500 g of water and 320 g of acrylic acid (Solution 2)

and a solution comprising 60 g of sodium hydroxide and 180 g of water (Solution 3).

63 g of polyethylene oxide (molecular mass of 20,000 g/mol) and 37.8 g of N,N-dimethyl-N-(2-ethyl methacrylate)-N-(3-sulfopropyl)ammonium sulfobetaine are dissolved in the sodium hydroxide solution at 65° C. contained in the reactor. Solution 1 is introduced continuously over 150 minutes, Solution 2 over 120 minutes and Solution 3 over 120 minutes.

Once the introductions are complete (150 minutes), the temperature is increased over 30 minutes to 80° C. and a solution of 0.32 g of ammonium persulfate in 10 g of water and a solution of 0.16 g of sodium bisulfite in 10 g of water are then added.

The reaction mixture is then left stirring for 120 minutes at 80° C.

The mixture is finally cooled to room temperature.

A solution of grafted copolymer is obtained.

### EXAMPLE 6

Grafted copolymer consisting of a POE-POP diblock trunk and of acrylic acid polymer grafts

10 g of sodium hydroxide are dissolved in 462 g of water, at room temperature, in a 2-liter glass reactor fitted with a jacket and a stirring system (120 rev/min). The temperature is then increased to 65° C.

The following are prepared:

an initiator solution comprising 0.46 g of ammonium persulfate and 10 g of water (Solution 1)

a solution comprising 28 g of water and 92 g of acrylic acid (Solution 2)

and a solution comprising 10 g of sodium hydroxide and 20 g of water (Solution 3).

18.2 g of a diblock copolymer of ethylene oxide and of propylene oxide (molecular mass 4150 g/mol, 50% by weight EO and 50% by weight PO) are dissolved in the sodium hydroxide solution at 65° C. contained in the reactor.

After Solution 1 has been introduced, in a single portion, into the reactor, Solutions 2 and 3 are introduced continuously over 300 minutes.

Once the introductions are complete, the reaction mixture is left stirring for 300 minutes at 65° C.

The mixture is finally cooled to room temperature.

A solution of grafted copolymer having a molecular mass of about 600,000 is obtained.

### EXAMPLE 7

Polyethylene oxide/polyacrylic acid block copolymer

—preparation of a polyoxyethylene xanthate disulfide—

100 g of polyoxyethylene (molecular mass 20,000 g/mol) and 300 g of toluene are placed as a stock solution in a 1-liter glass reactor fitted with a condenser and an anchor-type mechanical stirrer (180 rpm). The temperature of the reaction medium is brought to 35° C. Under a strong stream of nitrogen, 0.48 g of 60% NaH in oil are added over 30 min. Heating is maintained for 24 h. The reaction medium is then cooled to 30° C. 0.63 ml of CS₂ are then added dropwise to the reaction medium. The reaction is complete after 1 h of heating at 30° C.

A xanthate salt is obtained, which is extracted, using a dropping funnel, with twice 150 ml of water. The aqueous phases are placed in a 1-liter glass reactor fitted with a condenser and a mechanical stirrer (120 rpm). The temperature of the reaction medium is lowered to 5° C. 50 ml of an aqueous solution containing 3.42 g of ammonium persulfate are added to the reactor over 1 h. The oxidation is complete after 24 h at 5° C.

A xanthate disulfide is obtained, which is isolated by evaporation of the water under vacuum. —synthesis of the POE/PAA copolymer—

12 g of the polyoxyethylene xanthate disulfide prepared above and 485 g of water are placed as a stock solution in a 2-liter glass reactor fitted with a condenser and an anchor-type stirrer (180 rpm). The temperature of the reaction medium is brought to 65° C. 114 g of aqueous 12.2% NaOH solution and 150 g of aqueous 67% acrylic acid solution are added simultaneously to the reaction medium. The heating time is 2 h at 65° C.

A block copolymer is obtained whose molecular mass exceeds 1,000,000.

### EXAMPLE 8

Aqueous biocidal compositions for the biocidal treatment of hard surfaces are prepared using:

an ethoxylated alcohol containing on average 12 carbon atoms and 6 ethylene oxide units (SA)

triclosan (B)

an aqueous solution of a copolymer (C) prepared in Examples 1 to 7, and

water

in amounts such that the said aqueous compositions comprise

10 parts by dry weight of (SA)

0.3 part by dry weight of (B)

3 parts by dry weight of (C) per 100 parts by weight of aqueous composition.

### EXAMPLE 9

Aqueous biocidal compositions for washing up by hand are prepared by using:

25 parts by weight (expressed as solids) of lauryl ether sulfate

10 parts by weight (expressed as solids) of sodium paraffin sulfonate

5 parts by weight of sodium cocoamphoacetate

0.2 part by weight of triclosan (B)

1 part by weight (expressed as solids) of an aqueous solution of a copolymer (C) prepared in Examples 1 to 7,

and water to obtain 100 parts of composition.

1. Process for the biocidal treatment of cutaneous, keratinous or textile surfaces, as well as of hard industrial, domestic or community surfaces, by applying to the said surfaces an aqueous biocidal composition comprising:
at least one biocidal agent (B)
at least one surfactant (SA) when the said biocide is hydrophobic, and
at least one water-soluble or water-dispersible organic copolymer (C) comprising
at least one oligomeric or macromolecular unit (D) which can interact with the said biocide or with the micelles of surfactant(s) containing the said biocide when the latter is hydrophobic, and
at least one hydrophilic macromolecular unit (E) which can interact with the surface to be treated and optionally with the said biocide.

2. Process according to claim 1, characterized in that the said copolymer (C) has a molecular mass of about 10,000 to 10,000,000, preferably of about 50,000 to 2,000,000.

3. Process according to claim 1 or 2, characterized in that the said copolymer (C) is
a block copolymer in which each unit (D) or (E) represents a block,
or a grafted copolymer consisting
of a trunk comprising an oligomeric or macromolecular unit (D) and of one or more grafts comprising a macromolecular unit (E)
or alternatively of a trunk comprising a macromolecular unit (E) and of one or more grafts comprising an oligomeric or macromolecular unit (D).

4. Process according to any one of claims 1 to 3, characterized in that the oligomeric or macromolecular units constituting the unit(s) (D) are chosen from those derived from
oxyalkylene oligomers or polymers (D1), in which the oxyalkylene repeating units are the same or different, the alkylene residue being linear or branched and containing from 2 to 4 carbon atoms, the said oligomers or polymers having an overall degree of polymerization of 2 to 3000, preferably of 5 to 500,
vinylpyrrolidone polymers or copolymers (D2) having a molecular mass of about 5000 to 1,000,000, preferably of 5000 to 400,000, optionally containing up to 50 mol % of units derived from at least one comonomer such as N-vinylimidazole or vinyl acetate,
polyvinyl alcohols (D3) having a molecular mass of about 5000 to 1,000,000, preferably of 5000 to 400,000.

5. Process according to claim 4, characterized in that the oxyalkylene oligomers or polymers (D1) are chosen from polyoxyethylene (POE) monoblocks, polyoxyethylene-polyoxypropylene (POE-POP) diblocks and (POE-POP-POE) triblocks.

6. Process according to any one of claims 1 to 5, characterized in that the hydrophilic unit (E) is chosen from those derived from:
homopolymers or copolymers (E1) obtained from the polymerization of at least one anionic, amphoteric or cationic, water-soluble, ethylenically unsaturated monomer
copolymers (E2), which are preferably water-soluble, obtained from the copolymerization of at least one water-soluble, anionic, amphoteric or cationic, preferably anionic, ethylenically unsaturated monomer, and of at least one nonionic, hydrophilic, ethylenically unsaturated comonomer.

7. Process according to claim 6, characterized in that the said anionic water-soluble ethylenically unsaturated monomer is chosen from water-soluble ethylenically unsaturated carboxylic acids, in particular acrylic, methacrylic, fumaric, maleic or itaconic acid or anhydride, N-methacroylalanine, N-acryloylhydroxyglycine, or water-soluble salts thereof, sulfonated or phosphonated water-soluble ethylenically unsaturated monomers such as, in particular, sulfopropyl acrylate or water-soluble salts thereof, water-soluble styrene sulfonates, or vinylphosphonic acid and water-soluble salts thereof.

8. Process according to claim 6, characterized in that the said amphoteric water-soluble ethylenically unsaturated monomer is chosen from N,N-dimethyl-N-methacryloxyethyl-N-(3-sulfopropyl)ammonium sulfobetaine, methacrylic acid amidopropyldimethylammonium betaine, 1-vinyl-3-(3-sulfopropyl)imidazolidium betaine, and 2-vinylpyridinium sulfopropylbetaine.

9. Process according to claim 6, characterized in that the cationic water-soluble ethylenically unsaturated monomer is chosen from trimethylaminoethyl-acrylamide or -methacrylamide chloride or bromide, trimethylaminobutyl-acrylamide or -methacrylamide methylsulfate, trimethylaminopropyl methacrylate chloride and 1-ethyl-2-vinylpyridinium bromide, chloride or methylsulfate.

10. Process according to claim 6, characterized in that the said nonionic comonomer is chosen from hydroxyethyl acrylate, hydroxyethyl methacrylate, methoxyethyl acrylate, acrylamide, N-dimethylaminomethyl methacrylate, methyl or ethyl acrylate or methacrylate, vinyl acetate, methyl or ethyl vinyl ether, N-vinylpyrrolidone, styrene, vinyl chloride and acrylonitrile, the amount of units derived from the said comonomer(s) representing up to 70%, preferably up to 50%, of the weight of the said copolymer (E2).

11. Process according to any one of claims 6 to 8, characterized in that the hydrophilic unit (E) is obtained from the polymerization of at least one water-soluble ethylenically unsaturated carboxylic acid (or a salt) and optionally of at least one water-soluble amphoteric ethylenically unsaturated monomer, the amount of amphoteric ethylenically unsaturated monomer representing from 1 to 50% and preferably from about 2 to 20%, of the total weight of ethylenically unsaturated monomers.

12. Process according to any one of claims 1 to 11, characterized in that the hydrophilic unit (E) has a molecular mass of greater than 10,000 and up to 10,000,000.

13. Process according to any one of claims 1 to 12, characterized in that the relative amounts of unit(s) (D) and of unit(s) (E) correspond to a ratio of the total mass of units (D)/total mass of units (E) of about 5/100 to 50/100, preferably of about 10/100 to 30/100.

14. Process according to any one of claims 1 to 13, characterized in that the copolymer (C) is chosen from:
grafted copolymers with a molecular mass of about 10⁴ to 10⁷, having a trunk consisting of a polyoxyethylene block with a molecular mass of about 10² to 2×10⁵, and of grafts consisting of polyacrylic acid,
grafted copolymers with a molecular mass of about 10⁴ to 10⁷, having a trunk consisting of a polyoxyethylene-polyoxypropylene diblock with a molecular mass of about 10² to 2×10⁵, and of grafts consisting of polyacrylic acid,
grafted copolymers with a molecular mass of about 10⁴ to 10⁷, having a trunk consisting of a polyoxyethylene-polyoxypropylene diblock with a molecular mass of about 10² to 2×10⁵, and of grafts consisting of random units derived from acrylic acid and from hydroxyethyl acrylate,
grafted copolymers with a molecular mass of about 10⁴ to 10⁷, having a trunk consisting of a polyoxyethylene block with a molecular mass of about 10² to 2×10⁵, and of grafts consisting of random units derived from acrylic acid and from N,N-dimethyl-N-(2-ethyl methacrylate)-N-(3-sulfopropyl)ammonium sulfobetaine,
grafted copolymers with a molecular mass of about 10⁴ to 10⁷, having a trunk consisting of a polyvinylpyrrolidone block with a molecular mass of about 5×10³ to 10⁶, and of grafts consisting of polyacrylic acid,
grafted copolymers with a molecular mass of about 10⁴ to 10⁷ having a trunk consisting of a random copolymer unit containing acrylic acid and methacrylate units, and of grafts consisting of methoxy polyethylene glycol units with a molecular mass of about 10² to 2×10⁵.

15. Process according to any one of claims 1 to 14, characterized in that the biocidal agent (B) is hydrophobic and chosen from
para-chloro-meta-xylenol or dichloro-meta-xylenol
4-chloro-m-cresol
resorcinol monoacetate
mono- or poly-alkyl or -aryl phenols, cresols or resorcinols, such as o-phenylphenol, p-tert-butyl-phenol or 6-n-amyl-m-cresol
alkyl and/or aryl-chloro- or -bromophenols, such as o-benzyl-p-chlorophenol
halogenated diphenyl ethers such as 2′,4,4′-trichloro-2-hydroxy-diphenyl ether (triclosan) and 2,2′-dihydroxy-5,5′-dibromo-diphenyl ether
chlorophenesin (p-chloro-phenylglyceric ether).

16. Process according to any one of claims 1 to 14, characterized in that the biocidal agent (B) is hydrophilic and chosen from
cationic biocides such as
quaternary monoammonium salts such as cocoalkyl-benzyldimethylammonium, (C₁₂-C₁₄) alkylbenzyldimethyl ammonium, cocoalkyldichlorobenzyldimethylammonium, tetradecylbenzyldimethylammonium, didecyldimethylammonium or dioctyldimethylammonium chlorides, myristyltrimethylammonium or cetyltrimethylammonium bromides
monoquaternary heterocyclic amine salts such as laurylpyridinium, cetylpyridinium or (C₁₂-C₁₄)alkylbenzylimidazolium chlorides
triphenylphosphonium fatty alkyl salts such as myristyltriphenylphosphonium bromide
polymeric cationic biocides, such as those derived from the reaction
of epichlorohydrin and of dimethylamine or of diethylamine
of epichlorohydrin and of imidazole
of 1,3-dichloro-2-propanol and of dimethylamine
of 1,3-dichloro-2-propanol and of 1,3-bis(dimethylamino)-2-propanol
of ethylene dichloride and of 1,3-bis(dimethylamino)-2-propanol
of bis(2-chloroethyl) ether and of N,N′-bis(dimethylaminopropyl)-urea or -thiourea
biguanidine polymeric hydrochlorides, such as Vantocil IB
amphoteric biocides such as derivatives of N-(N′-C₈-C₁₈alkyl-3-aminopropyl)glycine, of N-(N′-(N″-C₈-C₁₈-alkyl-2-aminoethyl)-2-aminoethyl)glycine, of N,N-bis (N′-C₈-C₁₈alkyl-2-aminoethyl)glycine, such as (dodecyl)(aminopropyl)glycine and (dodecyl)(diethylenediamine) glycine
amines such as N-(3-aminopropyl)-N-dodecyl-1,3-propanediamine
halogenated biocides, for instance iodophores and hypochlorite salts, such as sodium dichloroisocyanurate
phenolic biocides such as phenol, resorcinol and cresols.

17. Process according to any one of claims 1 to 15, characterized in that the surfactant present is nonionic, anionic, amphoteric or zwitterionic.

18. Process according to any one of claims 1 to 15 or according to claim 17, characterized in that the biocide (B) is hydrophobic and in that the said system (B) contains an agent for solubilizing the said hydrophobic biocide in water.

19. Process according to any one of claims 1 to 18, characterized in that the said biocidal composition contains
from about 0.01 to 20%, preferably from about 0.03 to 5%, of its weight of at least one biocidal agent (B)
from about 0.1 to 50%, preferably from about 0.5 to 30%, of its weight of at least one surfactant (SA), when the said biocidal agent is hydrophobic, and
from about 0.005 to 20%, preferably from about 0.05 to 10%, most particularly from 0.05 to 5%, of its weight of at least one copolymer (C).

20. Process according to any one of claims 1 to 19, characterized in that the said composition is diluted from 1 to 100 times, preferably from 1 to 1000 times, before use.

21. Process according to any one of claims 1 to 20, characterized in that the amount of aqueous biocidal composition used corresponds to a deposition of biocidal agent (B) of 0.01 to 10 g, preferably of 0.1 to 1 g, per m² of surface and to a deposition of copolymer (C) of 0.001 to 2 g, preferably of 0.01 to 0.5 g, per m² of surface.

22. Use, in an aqueous biocidal composition for the biocidal treatment of cutaneous, keratinous or textile surfaces, as well as of hard industrial, domestic or community surfaces, containing at least one biocidal agent (B) and at least one surfactant (SA) when the said biocidal agent is hydrophobic, of at least one water-soluble or water-dispersible organic copolymer (C) as an agent for the vectorization and/or controlled release of the said biocide(s) onto the hard surface to be treated, the said copolymer (C) comprising
at least one oligomeric or macromolecular unit (D) which can interact with the said biocide or with the micelles of surfactant(s) containing the said biocide when the latter is hydrophobic, and
at least one hydrophilic macromolecular unit (E) which can interact with the surface to be treated and optionally with the said biocide.

23. Use, according to claim 22, of the copolymer (C) described in any one of claims 2 to 14.

24. Use, according to claim 22 or 23, of the copolymer (C) in an aqueous biocidal composition comprising at least one biocide (B) described in claim 15 or 16.

25. Use, according to claim 24, of the copolymer (C) in an aqueous biocidal composition comprising at least one hydrophobic biocide (B) and at least one nonionic, anionic, amphoteric or zwitterionic surfactant.

26. Use, according to any one of claims 22 to 25, of the copolymer (C), in an amount such that the said aqueous biocidal composition contains
from about 0.01 to 20%, preferably from about 0.03 to 5%, of its weight of at least one biocidal agent (B)
from about 0.1 to 50%, preferably from about 0.5 to 30%, of its weight of at least one surfactant (SA), when the said biocidal agent is hydrophobic, and
from about 0.005 to 20%, preferably from about 0.05 to 10%, most particularly from 0.05 to 5%, of its weight of copolymer (C).

27. Process according to any one of claims 1 to 21 or use according to one of claims 22 to 26, characterized in that the said aqueous biocidal composition is a body hygiene composition for the hair or the skin, such as shampoos, lotions or gels for the hair or the body and liquid soaps for the face or the body.

28. Process according to any one of claims 1 to 21 or use according to one of claims 22 to 26, characterized in that the said aqueous biocidal composition is a detergent composition for handwashing laundry, for rinsing laundry or for washing up by hand.

29. Process according to any one of claims 1 to 21 or use according to one of claims 22 to 26, characterized in that the said aqueous biocidal composition is a detergent composition for cleaning and disinfecting hard industrial, domestic or community surfaces.

30. Aqueous biocidal composition containing
at least one noncationic biocidal agent (B)
at least one surfactant (SA) when the said biocide is hydrophobic, and
at least one water-soluble or water-dispersible organic copolymer (C) comprising
at least one oligomeric or macromolecular unit (D) which can interact with the said biocide or with the micelles of surfactant(s) containing the said biocide when the latter is hydrophobic, and
at least one hydrophilic macromolecular unit (E) which can interact with the surface to be treated and optionally with the said biocide.

31. Composition according to claim 30, characterized in that the said copolymer (C) is chosen from those described in any one of claims 2 to 14.

32. Composition according to claim 30 or 31, characterized in that the said biocide (B) is chosen from the noncationic biocides described in claim 15 or 16.

33. Composition according to any one of claims 30 to 32, characterized in that it comprises
from about 0.01 to 20%, preferably from about 0.03 to 5%, of its weight of at least one noncationic biocidal agent (B)
from about 0.1 to 50%, preferably from about 0.5 to 30%, of its weight of at least one surfactant (SA), when the said biocidal agent is hydrophobic, and
from about 0.005 to 20%, preferably from about 0.05 to 10%, most particularly from 0.05 to 5%, of its weight of copolymer (C).

34. Aqueous biocidal composition, characterized in that it comprises
at least one cationic biocidal agent (B)
at least one surfactant (SA) when the said biocide is hydrophobic, and
at least one water-soluble or water-dispersible organic copolymer (C) chosen from block or grafted copolymers comprising
at least one oligomeric or macromolecular unit (D) chosen from those derived from
oxyalkylene oligomers or polymers (D1) in which the oxyalkylene repeating units are the same or different, the alkylene residue being linear or branched and containing from 2 to 4 carbon atoms, the said oligomers or polymers having an overall degree of polymerization of 2 to 3000, preferably of 5 to 500
vinylpyrrolidone polymers or copolymers (D2) having a molecular mass of about 5000 to 1,000,000, preferably of 5000 to 400,000, optionally containing up to 50 mol % of units derived from at least one comonomer such as N-vinylimidazole or vinyl acetate
polyvinyl alcohols (D3) having a molecular mass of about 5000 to 1,000,000, preferably of 5000 to 400,000,
and at least one hydrophilic macromolecular unit (E) chosen from those derived from
homopolymers or copolymers (E1) obtained from the polymerization of at least one anionic, amphoteric or cationic, water-soluble, ethylenically unsaturated monomer
copolymers (E2), which are preferably water-soluble, obtained from the copolymerization of at least one water-soluble, anionic, amphoteric or cationic, preferably anionic, ethylenically unsaturated monomer and of at least one nonionic, hydrophilic, ethylenically unsaturated comonomer, each oligomeric or macromolecular unit (D) representing a block of the copolymer (C) when the latter is a block copolymer, or the trunk of the copolymer (C) when the latter is a grafted copolymer, and each hydrophilic macromolecular unit (E) representing a block of the copolymer (C) when the latter is a block copolymer, or the graft(s) of the copolymer (C) when the latter is a grafted copolymer.

35. Composition according to claim 34, characterized in that the said copolymer (C) has a molecular mass of about 10,000 to 10,000,000, preferably of about 50,000 to 2,000,000.

36. Composition according to claim 34 or 35, characterized in that the units (D) of the said copolymer (C) are chosen from polyoxyethylene (POE) monoblocks, polyoxyethylene-polyoxypropylene (POE-POP) diblocks and (POE-POP-POE) triblocks.

37. Composition according to any one of claims 34 to 36, characterized in that the units (E) of the said copolymer (C) are chosen from those derived from the monomers described in any one of claims 7 to 11.

38. Composition according to any one of claims 34 to 37, characterized in that the hydrophilic unit (E) has a molecular mass of greater than 10,000 and up to 10,000,000.

39. Composition according to any one of claims 34 to 38, characterized in that the relative amounts of unit(s) (D) and of unit(s) (E) correspond to a ratio of the total mass of units (D)/total mass of units (E) of about 5/100 to 50/100, preferably of about 10/100 to 30/100.

40. Composition according to any one of claims 34 to 39, characterized in that the copolymer (C) is chosen from:
grafted copolymers with a molecular mass of about 10⁴ to 10⁷, having a trunk consisting of a polyoxyethylene block with a molecular mass of about 10² to 2×10⁵, and of grafts consisting of polyacrylic acid,
grafted copolymers with a molecular mass of about 10⁴ to 10⁷, having a trunk consisting of a polyoxyethylene-polyoxypropylene diblock with a molecular mass of about 10² to 2×10⁵, and of grafts consisting of polyacrylic acid,
grafted copolymers with a molecular mass of about 10⁴ to 10⁷ having a trunk consisting of a polyoxyethylene-polyoxypropylene diblock with a molecular mass of about 10² to 2×10⁵, and of grafts consisting of random units derived from acrylic acid and from hydroxyethyl acrylate,
grafted copolymers with a molecular mass of about 10⁴ to 10⁷, having a trunk consisting of a polyoxyethylene block with a molecular mass of about 10² to 2×10⁵, and of grafts consisting of random units derived from acrylic acid and from N,N-dimethyl-N-(2-ethyl methacrylate)-N-(3-sulfopropyl)anmonium sulfobetaine,
grafted copolymers with a molecular mass of about 10⁴ to 10⁷, having a trunk consisting of a polyvinyl-pyrrolidone block with a molecular mass of about 5×10³ to 10⁶, and of grafts consisting of polyacrylic acid.
